(19) United States
(12) Patent Application Publication
(10) Pub. No.: US 2013/0213426 A1
(43) Pub. Date: Aug. 22, 2013

(54) TWO-STEP NAIL POLISH PRODUCT

(71) Applicants: Hy Si Bui, Piscataway, NJ (US); Luis Ortega, East Brunswick, NJ (US); Chunhua Li, Hillsborough, NJ (US); Sarah Fairneny, Garwood, NJ (US); Mohamed Kanji, Edison, NJ (US)

(72) Inventors: Hy Si Bui, Piscataway, NJ (US); Luis Ortega, East Brunswick, NJ (US); Chunhua Li, Hillsborough, NJ (US); Sarah Fairneny, Garwood, NJ (US); Mohamed Kanji, Edison, NJ (US)

(73) Assignee: L'OREAL, Paris (FR)

(21) Appl. No.: 13/882,644

(22) PCT Filed: Oct. 31, 2011
(86) PCT No.: PCT/US11/58518

§ 371 Date: Apr. 30, 2013

Related U.S. Application Data
(60) Provisional application No. 61/409,319, filed on Nov. 2, 2010.

(51) Int. Cl. A61K 8/86; A45D 29/12; A61K 8/84; A61Q 3/02; A61K 8/81

(52) U.S. Cl. CPC A61K 8/86; A61Q 3/02; A61K 8/8141; A61K 8/84; A45D 29/12
USPC 132/200; 424/61

(57) ABSTRACT

The present invention relates to a nail polish product, comprising: (1) a base coat composition containing: (i) at least one high gloss film forming agent chosen from a styrene maleic anhydride copolymer; (ii) at least one co-film forming agent chosen from an epoxy resin; (iii) at least one first solvent chosen from at least one volatile solvent and water; (iv) optionally, if water is employed as a first solvent, at least one auxiliary reactive agent chosen from a polyalkyleneamine or a combination of polyalkyleneamine and a polyurethane; (v) optionally, at least one colorant; and (vi) optionally, at least one plasticizer; and (2) a top coat composition containing: (i) at least one second solvent chosen from at least one volatile solvent and water; (ii) at least one main reactive agent chosen from at least one alkoxysilane comprising at least one solubilizing functional group and, if water is employed as a second solvent, at least one polyalkyleneamine or a combination of a polyalkyleneamine and a polyurethane; and (iii) optionally, at least one colorant, wherein the product does not require use of nitrocellulose and can be used to makeup or protect nails.

### CROSS-REFERENCE TO RELATED APPLICATION

This application claims the benefit of the filing date of U.S. Provisional Application No. 61/409,319 filed Nov. 2, 2010, the contents of which are incorporated by reference.

### FIELD OF THE INVENTION

The present invention relates to a nail polish product which does not require use of nitrocellulose. Such nail polish products are safer to manufacture, and have comparable or better adhesion properties than, traditional nail polish products.

### DISCUSSION OF THE BACKGROUND

Nail polish products traditionally contain a large amount of nitrocellulose, primarily because nitrocellulose provides good adhesion of the compositions to nails upon application. That is, nitrocellulose is the preferred adhesive agent for use in nail polish compositions, and constitutes the “gold standard” of adhesive agents in nail polish compositions. However, nitrocellulose has drawbacks, particularly with respect to consumer safety. Also, nail polish compositions containing nitrocellulose can have poor long wear characteristics. Further, nitrocellulose does not impart high gloss. As a result, alternatives to nitrocellulose-based nail polish compositions have been sought. Unfortunately, to date, such alternatives have been elusive, and commercial nail polish compositions typically contain large amounts of nitrocellulose.

There remains a need for nail polish products which are safe, glossy, and adhere well to nails and, ideally, contain minimal, if any, amounts of nitrocellulose.

### SUMMARY OF THE INVENTION

The present invention relates to a nail polish product comprising:
(a) at least one base coat composition containing:
   i.at least one high gloss film-forming agent chosen from a styrene maleic anhydride copolymer;
   ii.at least one co-film forming agent chosen from an epoxy resin;
   iii.at least one first solvent chosen from at least one volatile solvent and water;
   iv.optionally, if water is employed as a first solvent, at least one auxiliary reactive agent chosen from a polyalkyleneamine or a combination of a polyalkyleneamine and a polyurethane;
   v.optionally, at least one colorant; and
   vi.optionally, at least one plasticizer; and
(b) at least one top coat composition containing:
   i.at least one second solvent chosen from at least one volatile solvent and water;
   ii.at least one main reactive agent chosen from at least one alkoxysilane comprising at least one solubilizing functional group and, if water is employed as a second solvent, at least one polyalkyleneamine or a combination of a polyalkyleneamine and a polyurethane; and
   iii.optionally, at least one colorant, wherein the product does not require use of nitrocellulose.

The present invention further relates to methods for making up and/or protecting fingernails comprising topically applying the above-described nail polish product to the nails.

It has been surprisingly found by the inventors that the above-described product, when applied onto a finger nail, yields a nail polish coating having exceptional adhesion, long wear and high gloss, in the absence of nitrocellulose.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Volatile”, as used herein, means having a flash point of less than about 100° C.

“Transfer resistance” as used herein refers to the quality exhibited by products that are not readily removed by contact with another material, such as, for example, an item of clothing. Transfer resistance may be evaluated by any method known in the art for evaluating such. For example, transfer resistance of a composition may be evaluated by a modified “kiss” test. The modified “kiss” test may involve application of the composition to a fingernail followed by rubbing a material, for example, a sheet of paper, against the nail after expiration of a certain amount of time following application, such as 5 minutes after application. Similarly, transfer resistance of a composition may be evaluated by the amount of product transferred from a wearer to any other substrate, such as transfer from the nail of an individual to a sleeve when putting on clothing after the expiration of a certain amount of time following application of the composition to the nail. The amount of composition transferred to the substrate (e.g., sleeve or paper) may then be evaluated and compared. For example, a composition may be transfer resistant if a majority of the product is left on the wearer's nails. Further, the amount transferred may be compared with that transferred by other compositions, such as commercially available compositions. In a preferred embodiment of the present invention, little or no composition is transferred to the substrate from the nail.

“Long wear” products as used herein, refers to products where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to nails and evaluating the color of the composition after an extended period of time. For example, the color of a composition may be evaluated immediately following application to nails and these characteristics may then be re-evaluated and compared after a certain amount of time. Further, these characteristics may be evaluated with respect to other compositions, such as commercially available compositions.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The compositions and methods of the present invention can comprise, consist of, or consist essentially of the essential elements and limitations of the invention described herein, as well as any additional or optional ingredients, components, or limitations described herein or otherwise useful.

Base Coat Composition

According to the present invention, a base coat composition comprising at least one high gloss film forming agent chosen from a styrene maleic anhydride copolymer, is provided. “Styrene maleic anhydride copolymer,” as used herein, means any polymer obtained by copolymerization of one or more maleic anhydride comonomers and of one or more styrene comonomers, the maleic anhydride comonomers optionally being partially or completely hydrolysed.

In general, it is known that while styrene maleic anhydride copolymers provide high gloss, they have a tendency to be too brittle. Their use, therefore, has typically been limited to those applications where brittleness is not a concern. The inventors, however, have surprisingly found that styrene maleic anhydride copolymers, when mixed with certain softer co-film forming agents, may then be effectively employed in nail polish compositions in spite of their conventional brittle tendencies. It has been found that such combinations allow for a nail composition to be formulated, in the absence of nitrocellulose, having long wear, good adhesion and high gloss properties, above and beyond conventional nail polish compositions containing nitrocellulose.

According to preferred embodiments, the styrene maleic anhydride copolymer has a molar fraction of maleic anhydride units of between 0.1 and 0.95, more preferably between 0.4 and 0.9.

According to preferred embodiments, the styrene maleic anhydride copolymer has styrene and maleic anhydride monomers in a molar ratio of 1:3 to 3:1, more preferably in a molar ratio of 1:2 to 2:1, and more preferably in a molar ratio of about 1:1, including all ranges and subranges therebetween such as 1.2:1 and 1.4:1.

According to preferred embodiments, the styrene maleic anhydride copolymer has a weight-average molecular weight ranging from about 1,000 to 200,000, preferably from about 5,000 to 100,000, and most preferably from about 10,000 to 50,000.

According to preferred embodiments, the styrene maleic anhydride copolymer has a glass transition temperature (Tg) ranging from about 100° C. to 175° C., preferably from about 125° C. to 160° C., and more preferably from about 135° C. to 155° C.

Preferred styrene maleic anhydride copolymers for use in the base coat composition of the present invention include non-esterified styrene maleic anhydride copolymers. Suitable examples of non-esterified styrene maleic anhydride copolymers include, but are not limited to, Hercules products Scripset® 520 (styrene/maleic anhydride copolymer), as well as liquid solutions containing this polymer such as, for example, imPress® SC-700 (sodium solution) and imPress® SC-720 (ammonium solution); Atofina products such as the styrene/maleic anhydride (50/50) copolymer, in the form of an ammonium salt at 30% in water, sold under the reference SMA1000H® or the styrene/maleic anhydride (50/50) copolymer, in the form of a sodium salt at 40% in water, sold under the reference SMA1000HNa®; and Sartomer products such as SMA®1000 (styrene/maleic anhydride (50/50) copolymer having a Tg of 155° C., a Mn of 2000, and a Mw of 5500), SMA®2000 (styrene/maleic anhydride (2:1) copolymer having a Tg of 135° C., a Mn of 3000, and a Mw of 7500), and SMA®2021 (styrene/maleic anhydride (2:1) copolymer having a Tg of 155° C., a Mn of 12,000, and a Mw of 21,000).

The styrene maleic anhydride copolymers of the present invention may also be esterified. “Esterified styrene maleic anhydride copolymer” as used herein means a styrene maleic anhydride copolymer which has been esterified using a small alcohol compound. Preferably, the small alcohol compound has fewer than 8 carbon atoms, preferably fewer than five carbon atoms, and more preferably fewer than four carbon atoms. For example, a styrene maleic anhydride copolymer can be esterified via standard esterification techniques using butanol, isobutanol, propanol, isopropanol, ethanol, methanol or any mixture of these alcohols, to produce an esterified styrene maleic anhydride copolymer. It should be noted, however, that in the event an esterified styrene maleic anhydride copolymer is employed, it cannot be completely esterified. Preferred styrene maleic anhydride copolymers are those which are non-esterified.

In accordance with preferred embodiments, the at least one high gloss film-forming agent of the present invention is preferably present in the base coat composition in an amount of from about 1% to about 40% by weight, preferably from about 5% to about 30% by weight, and more preferably from about 10% to about 20% of the total weight of the composition, including all ranges and subranges therebetween, all weights based on the total weight of the base coat composition.

The base coat composition further comprises at least one co-film forming agent chosen from an epoxy resin. Particularly preferred epoxy resins include, but are not limited to, tosylamide epoxy resins such as those sold under the Polytex name by Estron Chemical, Inc. (for example, E-75, E-100 and NX-55). Epoxy resins have been shown to provide good adhesion and are less brittle than the high gloss film forming agents disclosed above.

Other co-film forming agents that may be employed in combination with an epoxy resin include, for example, radical polymers, polycondensates and polymers of natural origin.

Examples of suitable co-film forming agents include, but are not limited to, vinyl polymers such as, for example, polyvinyl butyral, acrylic (co)polymers, acrylic resins, styrene resins, acrylate-styrene resins, vinyl resins, vinyl copolymers, polyurethanes, polyesters, alkyd resins, cellulose polymers, such as nitrocellulose, cellulose esters, such as cellulose acetate, cellulose acetate propionate or cellulose acetate butyrate, resins resulting from the condensation of formaldehyde with an arylsulphonamide, and their mixtures. Other suitable co-film forming polymers may also include film formers which are more compatible with water. Examples of such film formers include, but are not limited to, starches and derivatives thereof, natural or synthetic gums and derivatives thereof, water soluble adhesives. Particularly preferred co-film forming agents are resin film forming agents, particularly polyester, acrylic and acrylic resins.

Specific examples of useful (meth)acrylic polymers or resins include, but are not limited to, copolymers of methyl methacrylate with butyl acrylate, butyl methacrylate, isobutyl methacrylate, or isobornyl methacrylate (e.g., PARALOID DM-55, PARALOID B48N, PARALOID B66, ELVACITE 2550), copolymers of isobutylmethacrylate and butyl methacrylate (e.g., ELVACITE 2046), and isobutyl methacrylate polymers (e.g., PARALOID B67).

Specific examples of polyester resins include, but are not limited to, polyester resins formed by reacting a polyhydric alcohol with a polybasic acid, e.g., phthalic acid such as, for example, UNIPLEX 670-P polyester resin, which is available from Unitex Chemical Corporation and which is a polyester resin obtained by reacting trimellitic acid, neopentyl glycol, and adipic acid.

As stated above, styrene maleic anhydride copolymers are known to be brittle. However, it has been surprisingly found that combining them with at least one co-film forming agent chosen from an epoxy resin effectively renders the styrene maleic anhydride copolymer less brittle, thereby allowing it to be incorporated into nail polish compositions having long wear, good adhesion and high gloss properties, without requiring the use of nitrocellulose. Accordingly, it is preferred that the at least one co-film forming agent chosen from an epoxy resin has a glass transition temperature (Tg) of less than about 100° C., preferably less than about 80° C.

According to preferred embodiments, the at least one co-film forming agent chosen from an epoxy resin is present in the compositions of the present invention in an amount ranging from about 0.1 to about 50% by weight, more preferably from about 1 to about 40% by weight, and most preferably from about 10 to about 30% by weight, including all ranges and subranges therebetween, all weights based on the total weight of the base coat composition.

According to preferred embodiments, the products of the present invention contain weight ratios of styrene maleic anhydride copolymer to at least one co-film forming agent chosen from an epoxy resin ranging from about 1 to 1, preferably from about 2 to 1, and more preferably from about 3 to about 1, including all ranges and subranges therebetween.

According to yet other preferred embodiments, the products of the present invention contain weight ratios of styrene maleic anhydride copolymer to at least one co-film forming agent chosen from an epoxy resin in a range of about 1 to 1, preferably about 1 to 2, and more preferably about 1 to 3, including all ranges and subranges therebetween.

The base coat composition of the present invention also includes at least one first solvent chosen from at least one volatile solvent and water. Any volatile solvent typically found in nail polish compositions can be used. Suitable volatile solvents include, but are not limited to, organic solvents which are liquid at ambient temperature. Examples of suitable volatile solvents include, but are not limited to, ketones such as methyl ethyl ketone, methyl isobutyl ketone, diisobutyl ketone, isophorone, cyclohexanone or acetone; alcohols, such as ethanol, isopropanol, diacetone alcohol, 2-butoxyethanol or cyclohexanol; glycols, such as ethylene glycol, propylene glycol, pentylene glycol or glycerol; propylene glycol ethers, such as propylene glycol monomethyl ether, propylene glycol monomethyl ether acetate or dipropylene glycol mono(n-butyl) ether; short-chain esters (having a total of 2 to 7 carbon atoms), such as ethyl acetate, methyl acetate, propyl acetate, n-butyl acetate or isopentyl acetate; alkanes, such as decane, heptane, dodecane or cyclohexane; aldehydes, such as benzaldehyde or acetaldehyde; and their mixtures. Most preferred are short-chain esters (having a total of from 2 to 8 carbon atoms).

In accordance with preferred embodiments, the at least one first solvent, is preferably present in the composition in an amount of from about 1% to about 90% by weight, preferably from about 10% to about 80% by weight, preferably from about 30 to about 75% by weight, including all ranges and subranges therebetween, all weights based on the total weight of the base coat composition.

In the event that water is employed as a first solvent, the base coat composition may also contain at least one auxiliary reactive agent chosen from a polyalkyleneamine or a combination of a polyalkyleneamine and a polyurethane. Non-limiting examples of polyalkyleneamines include polyethyleneimines such as Lupasol® products commercially available from BASF. Suitable examples of Lupasol® polyethyleneimines include Lupasol® PS, Lupasol® PL, Lupasol® PR8515, Lupasol® G20, Lupasol® G35 as well as Lupasol® SC Polyethyleneimine Reaction Products (such as Lupasol® SC-61B, Lupasol® SC-62J, and Lupasol® SC-86X). Other non-limiting examples of polyethyleneimines which may be used in the composition according to the present invention are the Epomin® products commercially available from Aceto. Suitable examples of Epomin® polyethyleneimines include Epomin® SP-006, Epomin® SP-012, Epomin® SP-018, and Epomin® P-1000. These examples include substituted polyethyleneimines.

With respect to the polyurethane, the polyurethane may be commercially available as an aqueous dispersion. “Aqueous polyurethane dispersion” as used herein means the aqueous polyurethane dispersions disclosed in U.S. Pat. No. 7,445,770 and/or U.S. Pat. No. 7,452,770, the entire contents of both of which are hereby incorporated by reference.

More specifically, aqueous polyurethane dispersions are preferably the reaction products of:

A) a prepolymer according to the formula:

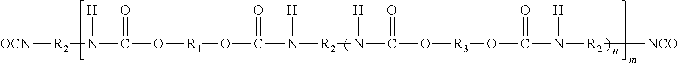

wherein R₁ represents a bivalent radical of a dihydroxyl functional compound, R₂ represents a hydrocarbon radical of an aliphatic or cycloaliphatic polyisocyanate, R₃ represents a radical of a low molecular weight diol, optionally substituted with ionic groups, n is from 0 to 5, and m is >1;

B) at least one chain extender according to the formula: H₂N—R₄—NH₂ wherein R₄ represents an alkylene or alkylene oxide radical not substituted with ionic or potentially ionic groups; and

C) at least one chain extender according to the formula: H₂N—R₅—NH₂ wherein R₅ represents an alkylene radical substituted with ionic or potentially ionic groups.

Suitable dihydroxyl compounds for providing the bivalent radical R₁ include those having two hydroxy groups and having number average molecular weights of from about 700 to about 16,000, and preferably from about 750 to about 5000. Examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. The polyester polyols, polyether polyols and polyhydroxy polycarbonates are preferred. Mixtures of various such compounds are also within the scope of the present invention.

Suitable polyisocyanates for providing the hydrocarbon radical R₂ include organic diisocyanates having a molecular weight of from about 112 to 1,000, and preferably from about 140 to 400. Preferred diisocyanates are those represented by the general formula R₂(NC0)₂ indicated above in which R₂ represents a divalent aliphatic hydrocarbon group having from 4 to 18 carbon atoms, a divalent cycloaliphatic hydrocarbon group having from 5 to 15 carbon atoms, a divalent araliphatic hydrocarbon group having from 7 to 15 carbon atoms or a divalent aromatic hydrocarbon group having 6-15 carbon atoms. Examples of the organic diisocyanates which are suitable include tetramethylene diisocyanate, 1,6-hexamethylene diisocyanate, dodecamethylene diisocyanate, cyclohexane-1,3- and -1,4-diisocyanate, 1-isocyanato-3-isocyanatomethyl-3,5,5-trimethylcyclohexane (isophorone diisocyanate or IPDI), bis-(4-isocyanatocyclohexyl)-methane, 1,3- and 1,4-bis(isocyanatomethyl)-cyclohexane, bis-(4-isocyanato-3-methyl-cyclohexyl)-methane, isomers of toluene diisocyanate (TDI) such as 2,4-diisocyanatotoluene, 2,6-diisocyanatotoluene, mixtures of these isomers, hydrogenated TDI, 4,4′-diisocyanato diphenyl methane and its isomeric mixtures with 2,4′- and optionally 2,2′-diisocyanato diphenylmethane, and 1,5-diisocyanato naphthalene. Mixtures of diisocyanates can, of course, be used. Preferred diisocyanates are aliphatic and cycloaliphatic diisocyanates. Particularly preferred are 1,6-hexamethylene diisocyanate and isophorone diisocyanate.

“Low molecular weight diols” in the context of R₃ means diols having a molecular weight from about 62 to 700, preferably 62 to 200. They may contain aliphatic, alicyclic or aromatic groups. Preferred compounds contain only aliphatic groups. The low molecular weight diols having up to about 20 carbon atoms per molecule include ethylene glycol, diethylene glycol, propane 1,2-diol, propane 1,3-diol, butane 1,4-diol, butylene 1,3-glycol, neopentyl glycol, butyl ethyl propane diol, cyclohexane diol, 1,4-cyclohexane dimethanol, hexane 1,6-diol, bisphenol A (2,2-bis(4-hydroxyphenyl)propane), hydrogenated bisphenol A (2,2-bis(4-hydroxycyclohexyl)propane), and mixtures thereof. Optionally, the low molecular weight diols may contain ionic or potentially ionic groups. Suitable lower molecular weight diols containing ionic or potentially ionic groups are those disclosed in U.S. Pat. No. 3,412,054, the contents of which is hereby incorporated by reference. Preferred compounds include dimethylol butanoic acid (DMBA), dimethylol propionic acid (DMBA) and carboxyl-containing caprolactone polyester diol. If lower molecular weight diols containing ionic or potentially ionic groups are used, they are preferably used in an amount such that <0.30 meq of COOH per gram of polyurethane in the polyurethane dispersion are present.

The prepolymer is chain extended using two classes of chain extenders. First, compounds having the formula: H₂N—R₄—NH₂ wherein R₄ represents an alkylene or alkylene oxide radical not substituted with ionic or potentially ionic groups. Alkylene diamines include hydrazine, ethylenediamine, propylenediamine, 1,4-butylenediamine and piperazine. The alkylene oxide diamines include 3-{2-[2-(3-aminopropoxy)ethoxy]ethoxy}propylamine (also known as dipropylamine diethyleneglycol or DPA-DEG available from Tomah Products, Milton, Wis.), 2-methyl-1,5-pentanediamine (Dytec A from DuPont), hexane diamine, isophorone diamine, and 4,4-methylenedi-(cyclohexylamine), and the DPA-series ether amines available from Tomah Products, Milton, Wis., including dipropylamine propyleneglycol, dipropylamine dipropyleneglycol, dipropylamine tripropyleneglycol, dipropylamine poly(propylene glycol), dipropylamine ethyleneglycol, dipropylamine poly(ethylene glycol), dipropylamine 1,3-propane diol, dipropylamine 2-methyl-1,3-propane diol, dipropylamine 1,4-butane diol, dipropylamine 1,3-butane diol, dipropylamine 1,6-hexane diol and dipropylamine cyclohexane-1,4-dimethanol. Mixtures of the listed diamines may also be used.

The second class of chain extenders are compounds having the formula: H₂N—R₅—NH₂ wherein R₅ represents an alkylene radical substituted with ionic or potentially ionic groups. Such compounds have an ionic or potentially ionic group and two groups that are reactive with isocyanate groups. Such compounds contain two isocyanate-reactive groups and an ionic group or group capable of forming an ionic group. The ionic group or potentially ionic group can be selected from the group consisting of ternary or quaternary ammonium groups, groups convertible into such a group, a carboxyl group, a carboxylate group, a sulfonic acid group and a sulfonate group. The at least partial conversion of the groups convertible into salt groups of the type mentioned may take place before or during the mixing with water. Specific compounds include diaminosulfonates, such as for example the sodium salt of N-(2-aminoethyl)-2-aminoethane sulfonic acid (AAS) or the sodium salt of N-(2-aminoethyl)-2-aminopropionic acid.

The polyurethane according to the present invention may also include compounds which are situated in each case at the chain ends and terminate said chains (chain terminators) as described in U.S. Pat. No. 7,445,770 and/or U.S. Pat. No. 7,452,770.

Preferably, the polyurethane may also be employed in the compositions of the present invention in the form of an aqueous dispersion, wherein the aqueous polyurethane dispersion has a viscosity of less than 2000 mPa·s at 23 C, preferably less than 1500, preferably less than 1000, including all ranges and subranges therebetween. Further preferably, the aqueous polyurethane dispersion has a glass transition temperature below 0° C.

Also preferably, the aqueous polyurethane dispersion has a solids content based on the weight of the dispersion of from 20% to 60%, preferably from 25% to 55% and preferably from 30% to 50%, including all ranges and subranges therebetween.

Suitable polyurethane compounds for use in the present invention include those available as, but not limited to, aqueous polyurethane dispersions sold under the BAYCUSAN® name by Bayer such as, for example, BAYCUSAN® C1000 (polyurethane-34), BAYCUSAN® C1001 (polyurethane-34), BAYCUSAN® C1003 (polyurethane-32), and BAYCUSAN® C1004 (polyurethane-35)

The at least one auxiliary reactive agent may be employed in the composition of the invention in an amount ranging from about 0.01 to about 3% by weight, such as from about 0.05 to about 1% by weight, and from about 0.1 to about 0.5% by weight, based on the total weight of the base coat composition.

According to particularly preferred embodiments of the present application, the base coat composition may further comprise at least one colorant. Any colorant typically found in nail polish compositions can be used. Suitable colorants include, but are not limited to, lipophilic dyes, pigments and pearlescent agents, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

In accordance with preferred embodiments, the colorant, if present, is preferably employed in the composition in an amount of from about 0.01% to about 20% by weight, preferably from about 0.1% to about 15% by weight, preferably from about 0.5 to about 10% by weight, including all ranges and subranges therebetween, all weights based on the total weight of the base coat composition.

It may also be desirable to employ a plasticizer in the compositions of the present invention. Any plasticizing agent typically found in nail polish compositions can be used. Examples of suitable plasticizers include, but are not limited to, glycols and their ether or ester derivatives, esters of acids, in particular carboxylic acids, such as citrates, adipates, carbonates, tartrates, phosphates or sebacates, oxyethylenated derivatives, such as oxyethylenated oils, and their mixtures. For example, suitable plasticizing agents include, but are not limited to, tributyl phosphate, tributoxyethyl phosphate, tricresyl phosphate, triphenyl phosphate, glycerol triacetate, butyl stearate, butyl glycolate, benzyl benzoate, butyl acetyltricinoleate, glyceryl acetyltricinoleate, dibutyl phthalate, diisobutyl phthalate, dioctyl phthalate, dimethoxyethyl phthalate, diamyl phthalate, triethyl citrate, tributyl citrate, tributyl acetylcitrate, tri(2-ethylhexyl)acetylcitrate, dibutyl tartrate, camphor, and mixtures thereof.

In accordance with preferred embodiments, the plasticizer, is preferably present in the composition in an amount of from about 0.01% to about 25% by weight, preferably from about 0.1% to about 22% by weight, preferably from about 1 to about 20% by weight, including all ranges and subranges therebetween, all weights based on the total weight of the base coat composition.

TOP COAT COMPOSITION According to the present invention, the top coat composition of the present invention contains at least one second solvent chosen from at least one volatile solvent and water. Suitable volatile solvents include those that have already been described above.

In accordance with preferred embodiments, the second solvent is preferably present in the composition in an amount of from about 1% to about 90% by weight, preferably from about 10% to about 80% by weight, preferably from about 30 to about 75% by weight, including all ranges and subranges therebetween, all weights based on the total weight of the top coat composition

The top coat composition also comprises at least main reactive agent chosen from an alkoxysilane comprising at least one solubilizing functional group and, if water is employed as a second solvent, a polyalkyleneamine or a combination of polyalkyleneamine and polyurethane.

With respect to the alkoxysilane, the term “at least one solubilizing functional group” means any functional chemical group facilitating the bringing into solution of the alkoxysilane in the solvent or in a combination of solvents of the composition, for example, in solvents chosen from water and water-alcoholic mixtures.

Suitable solubilizing functional groups for use in accordance with the present disclosure include, but are not limited to, primary, secondary, and tertiary amine, aromatic amine, alcohol, carboxylic acid, sulfonic acid, anhydride, carbamate, urea, guanidine, aldehyde, ester, amide, epoxy, pyrrole, dihydroimidazole, gluconamide, pyridyle, and polyether groups.

The at least one alkoxysilane present in the composition comprises at least one solubilizing functional group, which may be identical or different, such as those previously defined.

The at least one alkoxysilane comprising at least one solubilizing functional group present in the composition of the present disclosure may comprise at least one silicon atom, for example, one silicon atom.

The at least one alkoxysilane comprising at least one solubilizing functional group present in the composition may, in at least one embodiment, comprise two or three alkoxy functions. In another embodiment, the alkoxy functional groups are chosen from methoxy and ethoxy functional groups.

According to a further embodiment, the at least one alkoxysilane comprising at least one solubilizing functional group present in the composition of the present disclosure is chosen from compounds of formula (I):

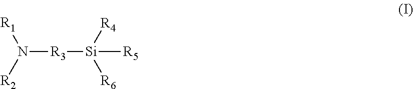

wherein:

R₄ is chosen from halogen atoms, OR′ groups, and R₁₁ groups;

R₅ is chosen from halogen atoms, OR″ groups, and R₁₂ groups;

R₆ is chosen from halogen atoms, OR′″ groups, and R₁₃ groups;

R₁, R₂, R₃, R′, R″, R′″, R₁₁, R₁₂, and R₁₃, which may be identical or different, are chosen from linear and branched, saturated and unsaturated hydrocarbon groups, optionally bearing at least one additional chemical group, wherein R₁, R₂, R′, R″, and R′″ may also be chosen from hydrogen; at least two groups R₄, R₅, and R₆ are different from R₁₁, R₁₂, and R₁₃, and at least two groups R′, R″, and R′″ are not hydrogen.

In at least one embodiment, the R₁, R₂, R′, R′₁, R′₂, R′₃, R″, and R′″ groups are chosen from C₁-C₁₂ alkyl, C₆-C₁₄ aryl, C₁-C₈ alkyl-C₆-C₁₄ aryl, and C₆-C₁₄ aryl-C₁-C₈-alkyl radicals.

According to a second embodiment of the present disclosure, the at least one alkoxysilane comprising at least one solubilizing functional group present in the composition is chosen from compounds of formula (II):

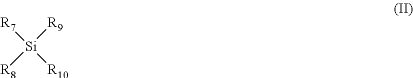

wherein:

R₉ is chosen from halogen atoms and OR′₉ groups and R₁₀ is chosen from halogen atoms and OR′₁₀ groups; wherein at least one of R₉ and R₁₀ is not a halogen;

R′₉ and R′₁₀, which may be identical or different, are chosen from hydrogen, and linear and branched, saturated and unsaturated C₁-C₁₄ hydrocarbon groups; wherein at least one of R₉ and R₁₀ is not hydrogen;

R₇ is a non hydrolyzable functional group providing a cosmetic effect, and

R₈ is a non hydrolyzable functional group bearing at least one function chosen from: amines, carboxylic acids and salts thereof, sulfonic acids and salts thereof, polyols such as glycol, polyethers such as polyalkylene ether, and phosphoric acids and salts thereof.

As used herein, the term “functional group providing a cosmetic effect” means a group derived from an entity chosen from reducing agents, oxidizing agents, coloring agents, polymers, surfactants, antibacterial agents, and UV absorbing filters.

In at least one embodiment, the functional group providing a cosmetic effect is a group derived from a coloring agent.

According to a third embodiment of the present disclosure, the at least one alkoxysilane comprising at least one solubilizing functional group present in the composition of the present disclosure is chosen from compounds of formula (III):

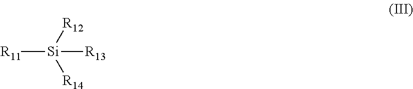

wherein:

R₁₂ is chosen from halogen atoms, OR′₁₂ groups, and Rₒ groups;

R₁₃ is chosen from halogen atoms, OR′₁₃ groups, and R′ₒ groups;

R₁₄ is chosen from halogen atoms, OR′₁₄ groups, and R″ₒ groups;

wherein at least two groups R₁₂, R_{1,3} and R₁₄ are different from Rₒ, R′ₒ, and R″ₒ groups;

R₁₁ is a group chosen from groups bearing at least one function chosen from: carboxylic acids and salts thereof, sulfonic acids and salts thereof, and polyalkylethers;

and Ro, R′o, R″o, R′₁₂, R′₁₃, and R′₁₄, which may be identical or different, are chosen from linear and branched, saturated and unsaturated, C₁-C₁₄ hydrocarbon groups optionally bearing at least one additional chemical functional group chosen from:

carboxylic acids and salts thereof, sulfonic acids and salts thereof, and polyalkylether functions, wherein R′₁₂, R′₁₃, and R₁₄ may also be chosen from hydrogen, and wherein at least two of the groups R′₁₂, R′₁₃, and R′₁₄ are not hydrogen.

In at least one embodiment, the R′₁₂, R′₁₃, R′₁₄, Rₒ, R′ₒ, and R″ₒ groups are chosen from C₁-C₁₂ alkyl groups, C₆-C₁₄ aryl groups, C₁-C₈ alkyl-C₆-C₁₄ aryl groups, and C₆-C₁₄ aryl-C₁-C₈ alkyl groups.

According to another embodiment of the present disclosure, the at least one alkoxysilane comprising at least one solubilizing functional group present in the composition of the present disclosure is chosen from compounds of formula (IV):

(R₂₁O)ₓ(R₂₂)_{y}Si-(A)ₚ-[NR₂₃-(A′)_{p′}]_{q}-[NR′₂₃-(A″)_{p″}]_{q″}]_{c′}-Si—(R′₂₂)_{y′}(OR′₂₁)_{x′} (IV)

wherein:

R₂₁, R₂₂, R′₂₁, and R′₂₂, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,

x is an integer ranging from 1 to 3,

y=3−x,

x′ is an integer ranging from 1 to 3,

y′=3−x′,

p=0 or 1,

p′=0 or 1,

p″=0 or 1,

q=0 or 1,

q′=0 or 1,

wherein at least one of q or q′ is not equal to zero,

A, A′, and A″, which may be identical or different, are chosen from linear and branched C1-C20 alkylene divalent radicals, and

R23 and R′23, which may be identical or different, are chosen from hydrogen and linear and branched, saturated and unsaturated hydrocarbon chains, optionally comprising at least one heteroatom, optionally interrupted by or substituted with at least one entity chosen from: ether, C1-C20 alcohol ester, amine, carboxyl, alkoxysilane, C6-C30 aryl, hydroxyl, and carbonyl groups, and aromatic, heterocyclic, and non-heterocyclic rings, optionally substituted with at least one group chosen from C3-C20 alcohol ester, amine, amide, carboxyl, alkoxysilane, hydroxyl, carbonyl, and acyl groups.

As defined above, R21, R22, R′21, and R′22, which may be identical or different, may be chosen from hydrocarbon chains. As used herein, the term “hydrocarbon chain” means, for example, a chain comprising from 1 to 10 carbon atoms.

Likewise, R23 and R′23 may be chosen from hydrocarbon chains. In such an embodiment, the hydrocarbon chains may comprise from 1 to 10 carbon atoms.

According to one embodiment, the aromatic ring comprises from 6 to 30 carbon atoms. In another embodiment, the aromatic ring is an optionally substituted phenyl radical.

In at least one embodiment, in formula (IV) above:

R21=R′21,

R22=R′22,

x=x′,

y=y′,

p=p′,

A=A′,

q=1, and

q′=0.

According to a further embodiment, the at least one alkoxysilane comprising at least one solubilizing functional group used according to the present disclosure may also have at least one of the following characteristics:

R21, R22, R′21, and R′22, which may be identical or different, are chosen from C1-C4 alkyl groups;

p=p′=1;

A and A′, which may be identical or different, are chosen from linear C1-C4 alkylene groups; and/or

R23 is hydrogen.

According to this embodiment, the at least one alkoxysilane comprising at least one solubilizing functional group may be chosen from compounds of formula (V):

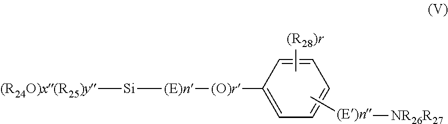

wherein:

R24 and R25, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,

x″=2 or 3,

y″=3−x″,

n″=0 or 1,

n″=0 or 1,

E and E′, which may be identical or different, are chosen from linear and branched C1-C20 alkylene divalent radicals,

R26 and R27, which may be identical or different, are chosen from hydrogen and linear and branched, saturated and unsaturated hydrocarbon chains, optionally comprising at least one heteroatom, optionally interrupted by or substituted with at least one entity chosen from: ether, C1-C20 alcohol ester, amine, carboxyl, alkoxysilane, C6-C30 aryl, hydroxyl, and carbonyl groups, and aromatic, heterocyclic, and non-heterocyclic rings, optionally substituted with at least one group chosen from: C1-C20 alcohol ester, amine, amide, carboxyl, alkoxysilane, hydroxyl, carbonyl, and acyl groups,

r is an integer ranging from 0 to 4,

r′=0 or 1, and

R28, which may be identical or different, is chosen from hydrogen and linear and branched, saturated and unsaturated hydrocarbon chains, comprising, for example, from 1 to 10 carbon atoms and optionally at least one heteroatom, optionally interrupted by or substituted with at least one entity chosen from: ether, C1-C20 alcohol ester, amine, carboxyl, alkoxysilane, C6-C30 aryl, hydroxyl, and carbonyl groups, and aromatic, heterocyclic, and non-heterocyclic rings, optionally substituted with at least one group chosen from: C1-C20 alcohol ester, amine, amide, carboxyl, alkoxysilane, hydroxyl, carbonyl, and acyl groups.

As defined above, R24 and R25, which may be identical or different, may be chosen from hydrocarbon chains. As used herein, the term “hydrocarbon chain” is intended to mean a chain comprising, for example, from 1 to 10 carbon atoms.

Likewise, R26 and R27 may be chosen from hydrocarbon chains. In this embodiment, the hydrocarbon chains may comprise from 1 to 10 carbon atoms.

According to another embodiment, the aromatic ring comprises from 6 to 30 carbon atoms. In a further embodiment, the aromatic ring is an optionally substituted phenyl radical.

According to at least one embodiment, the at least one alkoxysilane comprising at least one solubilizing functional group used in accordance with the present disclosure may have at least one of the following characteristics:

R24 is a C1-C4 alkyl group;

x″=3;

n′=n″=1

r=r′=0; and/or

R26 and R27, which may be identical or different, are chosen from hydrogen and groups chosen from C1-C4 alkyl groups, C1-C4 hydroxyalkyl groups, and C1-C4 aminoalkyl groups.

According to this embodiment, the at least one alkoxysilane comprising at least one solubilizing functional group may be chosen from:

3-(m-aminophenoxy)propyl trimethoxysilane, of formula:

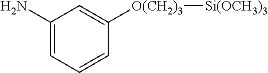

p-aminophenyl trimethoxysilane, of formula:

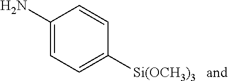

N-(2-aminoethylaminomethyl)phenethyl trimethoxysilane, of formula:

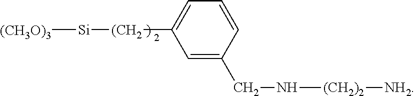

According to a further embodiment of the present disclosure, the at least one alkoxysilane comprising at least one solubilizing functional group present in the composition is chosen from compounds of formula (VI):

(R290)x1(R30)y1-Si-(A1)s-CH═O (VI)

wherein:

R29 and R30, which may be identical or different, are chosen from linear and branched, saturated and unsaturated hydrocarbon chains, optionally comprising at least one heteroatom, optionally interrupted by or substituted with at least one group chosen from ether, ester, amine, amide, carboxyl, hydroxyl, and carbonyl groups,

x1=2 or 3,

y1=3−x1,

Al is chosen from linear and branched C1-C20 alkylene divalent radicals, optionally interrupted by or substituted with at least one group chosen from C1-C30 alcohol ester, amine, carboxyl, alkoxysilane, C6-C30 aryl, hydroxyl, and carbonyl groups, and

s=0 or 1.

As defined above, R29 and R30, which may be identical or different can be chosen from hydrocarbon chains. As used herein, the term “hydrocarbon chain” means a chain comprising, for example, from 1 to 10 carbon atoms.

In another embodiment, the at least one alkoxysilane comprising at least one solubilizing functional group may have at least one of the following characteristics:

R29 and R30, which may be identical or different, are chosen from C1-C4, alkyl groups;

s=1; and

A1 is a linear C1-C4 alkylene group

According to this embodiment, the at least one alkoxysilane comprising at least one solubilizing functional group may be chosen from:

triethoxysilyl butyraldehyde, of formula:

(CH3CH2O)3-Si—(CH2)3-CH═O

triethoxysilyl undecanal, of formula:

(CH3CH2O)3-Si—(CH2)10-CH═O

and

triethoxysilyl undecanal, ethylene glycol acetal, of formula:

(CH3CH2O)3-Si—(CH2)10-CH(OCH2)2.

In a further embodiment, the at least one alkoxysilane comprising at least one solubilizing functional group is chosen from compounds of formula (VII):

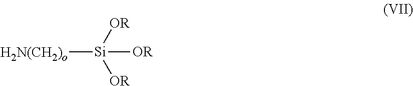

wherein the R radicals, which may be identical or different, are chosen from C1-C6 alkyl radicals and n is an integer ranging from 1 to 6, for example, from 2 to 4.

In at least one embodiment, the at least one alkoxysilane comprising at least one solubilizing functional group present in the composition of the present disclosure is a γ-aminopropyl triethoxysilane, also known as 3-aminopropyltriethoxysilane.

Particularly preferred alkoxysilanes having at least one solubilizing functional group include alkoxysilanes comprising a silicone atom. Suitable examples include those of formula R₍₄₋ₙ₎SiXₙ, wherein X is a hydrolysable group such as methoxy, ethoxy or 2-methoxyethoxy, R is a monovalent organic radical which contains 1 to 12 carbon atoms and may contain groups such as mercapto, epoxy, acrylyl, methacrylyl, amino or urea, and n is an integer from 1 to 4, and according to at least one embodiment is 3. Possible examples of useful alkoxysilanes include 3-mercaptopropyltriethoxysilane and aminoalkyltrialkoxysilanes such as 3-aminopropyltriethoxysilane, as described in French Patent Application No. FR 2 789 896.

Other useful alkoxysilanes are cited, for example, in Patent Application EP 1 216 022, which describes alkoxysilanes comprising at least one hydrocarbon chain containing a non-basic solubilizing chemical function. In this respect, non-limiting mention may be made of the HCl-neutralized sodium N-[(3-trimethoxysilyl)propyl]ethylenediaminetriacetate supplied by GELEST.

According to at least one embodiment, the alkoxysilanes may comprise at least one hydrocarbon chain containing fluorine atoms. Possible examples include but are not limited to the 3,3,3-trifluoropropyltriethoxysilane or tridecafluorooctyltriethoxysilane compounds described in Patent Application EP 1 510 197.

In another embodiment, the useful alkoxysilanes may be alkoxysilanes which carry a group having a cosmetic functional group, such as aromatic nitro dyes or anthraquinone, napthoquinone, benzoquinone, azo, xanthene, triarylmethane, azine, indoaniline, indophenolic or indoamine dyes; groups having a reductive effect, such as thiol groups, sulphinic acid or sulphinic salt, it being possible for these alkoxysilanes to carry a solubilizing non-hydrolysable group such as amino groups, carboxylic acids, sulphonic acids, sulphates, quaternary ammoniums, polyalcohols, polyether and phosphates. One possible example includes aminopropyl-N-(4,2-dinitrophenyl)aminopropyldiethoxysilane. Compounds of this kind are described, for example, in Patent Application EP 1 216 023.

The alkoxysilanes of the present disclosure may be amino aryl alkoxysilanes. Possible examples include but are not limited to the following compounds:

3-(m-aminophenoxy)propyltrimethoxysilane, of the formula:

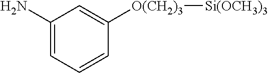

provided by GELEST,

p-aminophenyltrimethoxysilane, of formula:

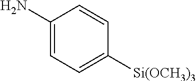

provided by GELEST, and

N-(2-aminoethylaminomethyl)phenethyltrimethoxysilane, of the formula:

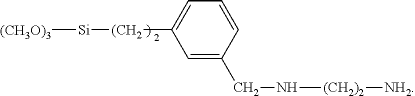

provided by GELEST.

According to at least one embodiment, the at least one organic silicon compound is N-(2-aminoethylaminomethyl)phenethyltrimethoxysilane.

The alkoxysilanes of the present disclosure may also be silanes having an aldehyde or acetal functional group, such as the triethoxysilylbutyraldehyde of formula (CH3CH₂O)₂Si(CH₂)₅CHO or the triethoxysilylunedecanol ethylene glycol acetal (CH₃CH₂O)₃Si(CH₂)₁₀CH(OCH₂)₂, which are provided by GELEST.

The alkoxysilanes may also be silanes containing non-primary amines, such as the bis[3-(triethoxysilyl)propyl]amine of the formula (CH₃CH₂O)₃—Si(CH₂)₃NH(CH₂)₃Si(OCH₂CH₃)₃ provided by Fluorochem, the bis[trimethoxysilylpropyl]amine of the formula (CH₃O)₃—Si(CH₂)₃NH(CH₂)₃Si(OCH₃)₃ provided by Gelest, the bis[methyldiethoxysilylpropyl]amine of the formula (CH₃CH₂O)₂CH₃Si(CH₂)₃NH(CH₂)₃SiCH₃(OCH₂CH₃)₂ provided by Gelest and the bis[3-trimethoxysilylpropyl]ethylenediamine of formula (CH₃O)₃Si(CH₂)₃NH(CH)₂NH(CH₂)₃Si(OCH₃)₃ provided by Gelest.

In another embodiment the at least one alkoxysilane is a trialkoxysilane comprising an amino substituent.

In at least one embodiment, the at least one alkoxysilane comprising at least one solubilizing functional group present in the composition of the present disclosure is a γ-aminopropyltriethoxysilane, also known as 3-aminopropyltriethoxysilane.

In the event that water is used as a second solvent, the at least one main reactive agent may also be chosen from a polyalkyleneamine or a combination of a polyalkyleneamine and a polyurethane.

Non-limiting examples of polyalkyleneamines which may be employed in the present invention include polyethyleneimines such as Lupasol® products commercially available from BASF. Suitable examples of Lupasol® polyethyleneimines include Lupasol® PS, Lupasol® PL, Lupasol® PR8515, Lupasol® G20, Lupasol® G35 as well as Lupasol® SC Polyethyleneimine Reaction Products (such as Lupasol® SC-61B, Lupasol® SC-62J, and Lupasol® SC-86X). Other non-limiting examples of polyethyleneimines which may be used in the composition according to the present invention are the Epomin® products commercially available from Aceto. Suitable examples of Epomin® polyethyleneimines include Epomin® SP-006, Epomin® SP-012, Epomin® SP-018, and Epomin® P-1000. These examples include substituted polyethyleneimines.

With respect to the polyurethane, the polyurethane may be commercially available as an aqueous dispersion. “Aqueous polyurethane dispersion” as used herein means the aqueous polyurethane dispersions disclosed in U.S. Pat. No. 7,445,770 and/or U.S. Pat. No. 7,452,770, the entire contents of both of which are hereby incorporated by reference.

More specifically, aqueous polyurethane dispersions are preferably the reaction products of:

A) a prepolymer according to the formula:

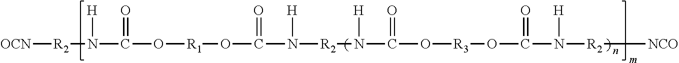

wherein R₁ represents a bivalent radical of a dihydroxyl functional compound, R₂ represents a hydrocarbon radical of an aliphatic or cycloaliphatic polyisocyanate, R₃ represents a radical of a low molecular weight diol, optionally substituted with ionic groups, n is from 0 to 5, and m is >1;

B) at least one chain extender according to the formula: H₂N—R₄—NH₂ wherein R₄ represents an alkylene or alkylene oxide radical not substituted with ionic or potentially ionic groups; and

C) at least one chain extender according to the formula: H₂N —R₅—NH₂ wherein R₅ represents an alkylene radical substituted with ionic or potentially ionic groups.

Suitable dihydroxyl compounds for providing the bivalent radical R₁ include those having two hydroxy groups and having number average molecular weights of from about 700 to about 16,000, and preferably from about 750 to about 5000. Examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. The polyester polyols, polyether polyols and polyhydroxy polycarbonates are preferred. Mixtures of various such compounds are also within the scope of the present invention.

Suitable polyisocyanates for providing the hydrocarbon radical R₂ include organic diisocyanates having a molecular weight of from about 112 to 1,000, and preferably from about 140 to 400. Preferred diisocyanates are those represented by the general formula R₂(NCO)₂ indicated above in which R₂ represents a divalent aliphatic hydrocarbon group having from 4 to 18 carbon atoms, a divalent cycloaliphatic hydrocarbon group having from 5 to 15 carbon atoms, a divalent araliphatic hydrocarbon group having from 7 to 15 carbon atoms or a divalent aromatic hydrocarbon group having 6-15 carbon atoms. Examples of the organic diisocyanates which are suitable include tetramethylene diisocyanate, 1,6-hexamethylene diisocyanate, dodecamethylene diisocyanate, cyclohexane-1,3- and -1,4-diisocyanate, 1-isocyanato-3-isocyanatomethyl-3,5,5-trimethylcyclohexane (isophorone diisocyanate or IPDI), bis-(4-isocyanatocyclohexyl)-methane, 1,3- and 1,4-bis(isocyanatomethyl)-cyclohexane, bis-(4-isocyanato-3-methyl-cyclohexyl)-methane, isomers of toluene diisocyanate (TDI) such as 2,4-diisocyanatotoluene, 2,6-diisocyanatotoluene, mixtures of these isomers, hydrogenated TDI, 4,4′-diisocyanato diphenyl methane and its isomeric mixtures with 2,4′- and optionally 2,2′-diisocyanato diphenylmethane, and 1,5-diisocyanato naphthalene. Mixtures of diisocyanates can, of course, be used. Preferred diisocyanates are aliphatic and cycloaliphatic diisocyanates. Particularly preferred are 1,6-hexamethylene diisocyanate and isophorone diisocyanate.

“Low molecular weight diols” in the context of R₃ means diols having a molecular weight from about 62 to 700, preferably 62 to 200. They may contain aliphatic, alicyclic or aromatic groups. Preferred compounds contain only aliphatic groups. The low molecular weight diols having up to about 20 carbon atoms per molecule include ethylene glycol, diethylene glycol, propane 1,2-diol, propane 1,3-diol, butane 1,4-diol, butylene 1,3-glycol, neopentyl glycol, butyl ethyl propane diol, cyclohexane diol, 1,4-cyclohexane dimethanol, hexane 1,6-diol, bisphenol A (2,2-bis(4-hydroxyphenyl)propane), hydrogenated bisphenol A (2,2-bis(4-hydroxycyclohexyl)propane), and mixtures thereof. Optionally, the low molecular weight diols may contain ionic or potentially ionic groups. Suitable lower molecular weight diols containing ionic or potentially ionic groups are those disclosed in U.S. Pat. No. 3,412,054, the contents of which is hereby incorporated by reference. Preferred compounds include dimethylol butanoic acid (DMBA), dimethylol propionic acid (DMBA) and carboxyl-containing caprolactone polyester diol. If lower molecular weight diols containing ionic or potentially ionic groups are used, they are preferably used in an amount such that <0.30 meq of COOH per gram of polyurethane in the polyurethane dispersion are present.

The prepolymer is chain extended using two classes of chain extenders. First, compounds having the formula: H₂N—R₄—NH₂ wherein R₄ represents an alkylene or alkylene oxide radical not substituted with ionic or potentially ionic groups. Alkylene diamines include hydrazine, ethylenediamine, propylenediamine, 1,4-butylenediamine and piperazine. The alkylene oxide diamines include 3-{2-[2-(3-aminopropoxy)ethoxy]ethoxy}propylamine (also known as dipropylamine diethyleneglycol or DPA-DEG available from Tomah Products, Milton, Wis.), 2-methyl-1,5-pentanediamine (Dytec A from DuPont), hexane diamine, isophorone diamine, and 4,4-methylenedi-(cyclohexylamine), and the DPA-series ether amines available from Tomah Products, Milton, Wis., including dipropylamine propyleneglycol, dipropylamine dipropyleneglycol, dipropylamine tripropyleneglycol, dipropylamine poly(propylene glycol), dipropylamine ethyleneglycol, dipropylamine poly(ethylene glycol), dipropylamine 1,3-propane diol, dipropylamine 2-methyl-1,3-propane diol, dipropylamine 1,4-butane diol, dipropylamine 1,3-butane diol, dipropylamine 1,6-hexane diol and dipropylamine cyclohexane-1,4-dimethanol. Mixtures of the listed diamines may also be used.

The second class of chain extenders are compounds having the formula: H₂N —R₅—NH₂ wherein R₅ represents an alkylene radical substituted with ionic or potentially ionic groups. Such compounds have an ionic or potentially ionic group and two groups that are reactive with isocyanate groups. Such compounds contain two isocyanate-reactive groups and an ionic group or group capable of forming an ionic group. The ionic group or potentially ionic group can be selected from the group consisting of ternary or quaternary ammonium groups, groups convertible into such a group, a carboxyl group, a carboxylate group, a sulfonic acid group and a sulfonate group. The at least partial conversion of the groups convertible into salt groups of the type mentioned may take place before or during the mixing with water. Specific compounds include diaminosulfonates, such as for example the sodium salt of N-(2-aminoethyl)-2-aminoethane sulfonic acid (AAS) or the sodium salt of N-(2-aminoethyl)-2-aminopropionic acid.

The polyurethane according to the present invention may also include compounds which are situated in each case at the chain ends and terminate said chains (chain terminators) as described in U.S. Pat. No. 7,445,770 and/or U.S. Pat. No. 7,452,770.

Preferably, the polyurethane may also be employed in the compositions of the present invention in the form of an aqueous dispersion, wherein the aqueous polyurethane dispersion has a viscosity of less than 2000 mPa·s at 23 C, preferably less than 1500, preferably less than 1000, including all ranges and subranges therebetween. Further preferably, the aqueous polyurethane dispersion has a glass transition temperature below 0° C.

Also preferably, the aqueous polyurethane dispersion has a solids content based on the weight of the dispersion of from 20% to 60%, preferably from 25% to 55% and preferably from 30% to 50%, including all ranges and subranges therebetween.

Suitable polyurethane compounds for use in the present invention include those available as, but not limited to, aqueous polyurethane dispersions sold under the BAYCUSAN® name by Bayer such as, for example, BAYCUSAN® C1000 (polyurethane-34), BAYCUSAN® C1001 (polyurethane-34), BAYCUSAN® C1003 (polyurethane-32), and BAYCUSAN® C1004 (polyurethane-35).

The at least main reactive agent is typically employed in the composition of the invention in an amount ranging from about 1 to about 90% by weight, such as from about 10 to about 80% by weight, and from about 30 to about % by weight, based on the total weight of the top coat composition.

According to particularly preferred embodiments of the present application, the top coat composition may further comprise at least one colorant. Any colorant typically found in nail polish compositions can be used. Suitable colorants include, but are not limited to, lipophilic dyes, pigments and pearlescent agents, and their mixtures.

Auxiliaries/Additives

The nail polish products of the present invention may additionally comprise an additive or auxiliary commonly used in cosmetic compositions and known to a person skilled in the art as being capable of being incorporated into a nail polish composition. Such additives or auxiliaries may be chosen from thickeners, coalescents, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

A person skilled in the art will take care to select the optional additional additives and/or the amounts thereof such that the advantageous properties of the product according to the invention are not, or are not substantially, adversely affected by the envisaged addition.

Needless to say, the composition of the invention should be cosmetically or dermatologically acceptable, i.e., it should contain non-toxic physiologically acceptable components. The composition may be in any galenic form normally employed in the cosmetic and dermatological fields which is suitable for topical administration onto nails.

One particularly preferred embodiment of the present invention is a product for application to nails which is substantially free of nitrocellulose (that is, contain up to 5% of nitrocellulose), essentially free of nitrocellulose (that is, contains up to 2% nitrocellulose), or free of nitrocellulose (that is, contains up to 0.25% nitrocellulose).

The inventors have found that when the styrene maleic anhydride copolymer in the base coat composition reacts with the at least one main reactive agent in the top coat, a resultant product having surprising and unexpected long-wear properties is formed.

Moreover, it has also been surprisingly found that the resultant product provides a degree of gloss that is at least comparable, and oftentimes higher, than that of conventional nitrocellulose-containing products.

According to preferred embodiments of the present invention, methods of making up or protecting nails comprising applying the product of the present invention to nails in an amount sufficient to makeup or protect the nails are provided. “Making up” as used herein means to provide decoration (for example, color) to the nail. “Protecting” as used herein means to inhibit damage to the nail (for example, chipping) by providing a protective layer on the nail. The application process involves first applying the base coat composition onto the fingernail and, once it is sufficiently dry, applying the top coat composition over top of the base coat composition.

The compositions according to the invention can be manufactured by known processes used generally in the cosmetics or dermatological field.

The following examples are intended to illustrate the invention without limiting the scope as a result. The percentages are given on a weight basis.

### EXAMPLES

### Example 1

### Base-Coat

| Phase | Chemical Name | Concentration (%) |
|---|---|---|
| A | Styrene Maleic | 15 |
| | Anhydride Copolymer | |
| | (SMA 2021) | |
| A | Ethyl Acetate | 28.25 |
| A | Propyl Acetate | 28.25 |
| A | Butyl Acetate | 11.5 |
| A | Tosylamide Epoxy | 12 |
| | Resin | |
| A | CAB Pigment | 5 |
| | Total | 100 |

### Example 1

### Top-Coat

| Phase | Chemical Name | Concentration (%) |
|---|---|---|
| A | Aminopropyltriethoxysilane | 50.00 |
| | (APTES) | |
| A | Isopropyl Alcohol | 50.00 |
| | Total | 100 |

### Example 2

### Base Coats

| Phase | Chemical name | % |
|---|---|---|
| A | SMA 1000P | 15 |
| A | ETHYL ACETATE | 29.5 |
| A | PROPYL ACETATE | 29.5 |
| A | BUTYL ACETATE | 9 |
| A | TOSYLAMIDE/EPOXY RESIN | 15 |
| | (75% resin in 25% Butyl acetate) | |
| A | Toyo Ink Red 7 | 2 |
| Total | | 100 |

### Example 2

### Top-coat

| | | EX2A | EX2B | EX2C | EX2D |
|---|---|---|---|---|---|
| Phase | Chemical Name | % | % | % | % |
| A | POLYETHYLENE | 10 | 20 | 30 | 40 |
| | IMINE | | | | |
| A | H2O | 90 | 80 | 70 | 60 |
| Total | | 100 | 100 | 100 | 100 |

### Example 3

### Base Coat

| Phase | Chemical Name | Concentration (%) |
|---|---|---|
| A | Styrene Maleic Anhydride | 15 |
| | Copolymer (SMA 2021) | |
| A | Ethyl Acetate | 28.25 |
| A | Propyl Acetate | 28.25 |
| A | Butyl Acetate | 11.5 |
| A | Tosylamide Epoxy Resin | 12 |
| A | CAB Pigment | 5 |
| | Total | 100 |

### Example 3

### Top Coat

| Phase | Chemical Name | Concentration (%) |
|---|---|---|
| A | Aminopropyltriethoxysilane | 30 |
| | (APTES) | |
| A | H2O | 70 |
| | Total | 100 |
| Phase | RM Name | Concentration (%) |
| A | Styrene Maleic | 17.78 |
| | Copolymer (SMA 2000 | |
| | Prep) | |
| A | Ethyl Acetate | 10.86 |
| A | Propyl Acetate | 21.21 |
| A | Butyl Acetate | 14.42 |
| A | Tosylamide Epoxy Resin | 12.23 |
| B | POLYETHYLENE IMINE | 0.25 |
| | (PEI; PM: 1700) | |
| B | Toyo Ink Red 7 | 3.0 |
| B | DI Water | 20.25 |
| | Total | 100 |

### Example 4

### Base-Coat

### Example 4

### Top-Coat

| Phase | Chemical Name | Concentration (%) |
|---|---|---|
| A | Aminopropyltriethoxy- | 50.00 |
| | silane (APTES) | |
| A | Isopropyl Alcohol | 50.00 |
| | Total | 100 |

What is claimed is:

1. A nail polish product, comprising:
(a) at least one base coat composition containing:
i. at least one high gloss film-forming agent chosen from a styrene maleic anhydride copolymer;
ii. at least one film forming agent chosen from an epoxy resin;
iii. at least one first solvent chosen from at least one volatile solvent and water;
iv. optionally, if water is employed as a first solvent, at least one auxiliary reactive agent chosen from a polyalkyleneamine or a combination of a polyalkyleneamine and a polyurethane;
v. optionally, at least one colorant; and
vi. optionally, at least one plasticizer; and
(b) at least one top coat composition containing:
i. at least one second solvent chosen from at least one volatile solvent and water;
ii. at least one main reactive agent chosen from at least one alkoxysilane comprising at least one solubilizing functional group and, if water is employed as a second solvent, at least one polyalkyleneamine or a combination of a polyalkyleneamine and a polyurethane; and
iii. optionally, at least one colorant, wherein the product does not require use of nitrocellulose.

2. The product of claim 1, wherein (a)(i) has a Tg ranging from about 100° C. to about 175° C.

3. The product of claim 1, wherein (a)(i) has a molecular weight ranging from about 1,000 to about 200,000.

4. The product of claim 1, wherein (a)(i) is present in an amount of from about 1% to about 40% by weight, based on the total weight of the base coat composition.

5. The product of claim 1, wherein (a) (ii) has a Tg of less than about 100° C.

6. The product of claim 1, wherein (a)(ii) is present in an amount of from about 0.1 to about 50% by weight, based on the total weight of the base coat composition.

7. The product of claim 1, wherein the weight ratio of (a) (i) to (a)(ii)is about 1 to 1.

8. The product of claim 1, wherein (a)(iii) and (b)(i) is chosen from ethyl acetate, propyl acetate, butyl acetate, water and mixtures thereof.

9. The product of claim 1, wherein (a)(iii) is present in an amount of from about 1 to about 90% by weight, based on the total weight of the base coat composition.

10. The product of claim 1, wherein (b)(i) is present in an amount of from about 1 to about 90% by weight, based on the total weight of the top coat composition.

11. The product of claim 1, wherein (a)(vi) is present in an amount of from about 0.01 to about 25% by weight, based on the total weight of the base coat composition.

12. The product of claim 1, wherein (b)(ii) includes at least one polyalkyleneamine.

13. The product of claim 1, wherein (b)(ii) includes at least one alkoxysilane comprising at least one solubilizing functional group.

14. The product of claim 13, wherein (b)(ii) is 3-aminopropyltriethoxysilane.

15. The product of claim 1 wherein (b)(ii) is a combination of a polyalkyleneamine and a polyurethane.

16. The product of claim 1, wherein (b)(ii) is present in an amount of from about 1 to about 90% by weight, based on the total weight of the top coat composition.

17. A method of making up finger nails comprising coating the nail with a two-step product comprising:
(a) a basecoat composition containing:
(i) at least one high gloss film-forming agent chosen from a styrene maleic anhydride copolymer;
(ii) at least one film forming agent chosen from an epoxy resin;
(iii) at least one first solvent chosen from at least one volatile solvent and water;
(iv) optionally, if water is employed as a first solvent, at least one auxiliary reactive agent chosen from a polyalkyleneamine or a combination of a polyalkyleneamine and a polyurethane;
(v) optionally, at least one colorant; and
(vi) optionally, at least one plasticizer; and
(b) a top coat composition containing:
(i) at least one second solvent chosen from at least one volatile solvent and water;
(ii) at least one main reactive agent chosen from at least one alkoxysilane comprising at least one solubilizing functional group and, if water is employed as a second solvent, at least one polyalkyleneamine or a combination of a polyalkyleneamine and a polyurethane; and
(iii) optionally, at least one colorant, wherein the product does not require use of nitrocellulose.

18. The method of claim 17, wherein (a)(i) has a Tg ranging from about 100° C. to about 175° C.

19. The method of claim 17, wherein (a)(i) has a molecular weight ranging from about 1,000 to about 200,000.

20. The method of claim 17, wherein (a)(i) is present in an amount of from about 1% to about 40% by weight, based on the total weight of the base coat composition.

21. The method of claim 17, wherein (a)(ii) has a Tg of less than about 100° C.

22. The method of claim 17, wherein (a)(ii) is present in an amount of from about 0.1 to about 50% by weight, based on the total weight of the base coat composition.

23. The method of claim 17, wherein the weight ratio of (a)(i) to (a)(ii) is about 1 to 1.

24. The method of claim 17, wherein (a)(iii) and (b)(i) is chosen from ethyl acetate, propyl acetate, butyl acetate, water and mixtures thereof.

25. The method of claim 17, wherein (a)(iii) is present in an amount of from about 1 to about 90% by weight, based on the total weight of the base coat composition.

26. The method of claim 17, wherein (b)(i) is present in an amount of from about 1 to about 90% by weight, based on the total weight of the top coat composition.

27. The method of claim 17, wherein (a)(vi) is present in an amount of from about 0.01 to about 25% by weight, based on the total weight of the base coat composition.

28. The method of claim 17, wherein (b)(ii) includes at least one polyalkyleneamine.

29. The method of claim 17, wherein (b)(ii) includes at least one alkoxysilane comprising at least one solubilizing functional group.

30. The method of claim 29, wherein (b)(ii) is 3-aminopropyltriethoxysilane.

31. The method of claim 17, wherein (b)(ii) is present in an amount of from about 1 to about 90% by weight, based on the total weight of the top coat composition.
